(19) United States
(12) Patent Application Publication
(10) Pub. No.: US 2025/0195135 A1
(43) Pub. Date: Jun. 19, 2025

(54) ELECTROPHYSIOLOGY CATHETER

(71) Applicant: St. Jude Medical, Cardiology Division, Inc., St. Paul, MN (US)

(72) Inventors: Zachary L. Helgeson, Richfield, MN (US); Dominic Leyden, St. Paul, MN (US); Neil D. Hawkinson, Ramsey, MN (US); Jodee M. Maes, Golden Valley, MN (US); Tobias Cajamarca, Shakopee, MN (US); Andrew Oliverius, Zimmerman, MN (US)

(21) Appl. No.: 18/843,906

(22) PCT Filed: Mar. 7, 2023
(86) PCT No.: PCT/US2023/014713

§ 371 Date: Sep. 4, 2024

Related U.S. Application Data
(60) Provisional application No. 63/317,612, filed on Mar. 8, 2022.

(51) Int. Cl. A61B 18/14; A61B 18/00

(52) U.S. Cl. CPC A61B 18/1492; A61B 2018/00577; A61B 2018/00642; A61B 2018/00773; A61B 2018/00839; A61B 2018/1407; A61B 2018/1467

(57) ABSTRACT

A catheter includes a multi-lumen catheter shaft that includes a proximal end, a loop portion, and a distal tip. A location system positioned in a first lumen of the shaft includes a distal shaft that extends along at least a portion of the loop, an electromagnetic sensor attached to an outer surface of the distal shaft at a location along the loop, and electromagnetic sensor wires extending proximally from the electromagnetic sensor. The electromagnetic sensor and the pair of electromagnetic sensor wires are positioned along a neutral axis of the multi-lumen catheter shaft. An electrode system includes first and second plurality of electrodes positioned along an outer surface of the loop, a first plurality of wires connected to the first plurality of electrodes and extending through the second lumen, and a second plurality of wires connected to the second plurality of electrodes and extending through the third lumen.

### CROSS-REFERENCE TO RELATED APPLICATION

This application claims the benefit of and priority to U.S. provisional application 63/317,612, titled “ELECTROPHYSIOLOGY CATHETER”, filed Mar. 8, 2022 the contents of which are incorporated by reference herein.

### BACKGROUND

The present invention relates generally to electrophysiology (EP) catheters and in particular to a multi-lumen catheter.

Tissue ablation may be used to treat a variety of clinical disorders. For example, tissue ablation may be used to treat cardiac arrhythmias by at least partially destroying (e.g., at least partially or completely ablating, interrupting, inhibiting, terminating conduction of, otherwise affecting, etc.) aberrant pathways that would otherwise conduct abnormal electrical signals to the heart muscle. Several ablation techniques have been developed, including cryoablation, microwave ablation, radio frequency (RF) ablation, pulsed field ablation, and high frequency ultrasound ablation. For cardiac applications, such techniques are typically performed by a clinician who introduces a catheter having an ablative tip to the endocardium via the venous vasculature. The clinician may utilize electrodes located at the distal end of the catheter to map electrocardiogram (ECG) signals within the heart and/or deliver ablative energy to selected regions within the heart. For example, pulsed field ablation utilizes high amplitude electrical pulses to cause electroporation of cell tissue, resulting in tissue ablation without inducement of tissue heating.

Successful electrophysiology procedures require precise knowledge regarding the location and orientation of the catheter within the heart. Localization/navigation systems include, for example, impedance-based systems and magnetic-based systems utilized to determine the location/orientation of the catheter within the patient. Impedance-based systems include a plurality of surface patch electrodes placed on the skin of a patient. Electrical signals are generated between the respective surface patch electrodes, and one or more electrodes located on the catheter measure an impedance based on the generated signals, wherein the impedance is utilized to generate an estimate of catheter location. Likewise, magnetic-based systems generate one or more magnetic fields in the proximity of the patient. A magnetic sensor or coil located on the catheter is utilized to measure the one or more magnetic fields and determine the position and/or orientation of the magnetic sensor based on the measured fields.

It would therefore be beneficial to develop an electrophysiology (EP) catheter capable of performing functions including mapping and ablation while providing accurate localization/orientation feedback to a clinician.

### SUMMARY

In some embodiments, a catheter includes a multi-lumen catheter shaft, a location system, and an electrode system. The multi-lumen catheter shaft includes a proximal end, a transition region, and a loop extending from a distal end of the transition region to a distal tip. The catheter shaft includes a first lumen, a second lumen, and a third lumen. The location system is positioned in the first lumen and includes a distal shaft that extends within the first lumen along at least a portion of the loop, an electromagnetic (EM) sensor attached to an outer surface of the distal shaft at a location along the loop, and a pair of electromagnetic (EM) sensor wires extending proximally from the EM sensor to a proximal end of the catheter shaft. The EM sensor and the pair of EM sensor wires are positioned along a neutral axis of the catheter shaft within the first lumen. The electrode system includes a first plurality of electrodes positioned along an outer surface of the loop, a second plurality of electrodes positioned along the outer surface of the loop, a first plurality of wires connected to the first plurality of electrodes and extending through the second lumen, and a second plurality of wires connected to the second plurality of electrodes and extending through the third lumen.

In another aspect, an electrophysiology catheter includes a multi-lumen shaft, a localization system, and an electrode system. The multi-lumen shaft includes a proximal end, a transition region, and a loop extending from a distal end of the transition region to a distal tip. The multi-lumen shaft includes a first lumen, a second lumen, and a third lumen. The localization system is positioned in the first lumen. The localization system includes an electromagnetic (EM) sensor and a pair of electromagnetic (EM) sensor wires extending proximally from the EM sensor. The electrode system is positioned in the first and second electrode lumens. The electrode system includes a first plurality of electrode wires extending through the second lumen and connected to a first plurality of electrodes located on an outer surface of the loop and a second plurality of electrode wires extending through the third lumen and connected to a second plurality of electrodes located on the outer surface of the loop. The localization system is electrically isolated from the electrode system by the walls of the multi-lumen shaft.

### DESCRIPTION OF THE DRAWINGS

FIG. 1A is a diagrammatic view of a medical system, according to some embodiments.

FIG. 1B is a diagrammatic view of an electrophysiology catheter configured to guide a loop carrying a plurality of electrodes into the heart according to some embodiments.

FIG. 2 is a side view of an electrophysiology catheter with a loop, according to some embodiments.

FIG. 3 is a cross-sectional view, taken at line 3-3 of FIG. 2, of a transition region of the catheter.

FIG. 4 is another cross-sectional view of the transition region of the catheter, taken at line 4-4 of FIG. 2.

FIG. 5 is an enlarged view of area 5 of FIG. 2 with a portion of the multi-lumen catheter shaft in the transition region of the catheter removed to show the localization system and the electrode system, according to some embodiments.

FIG. 6 is a cross-sectional view, taken at line 6-6 of FIG. 2, illustrating portions of the first and second lumens within the transition region.

FIG. 7 is a cross-sectional view of the loop, taken at line 7-7 of FIG. 2, illustrating an electrode recess and an electrode wire extending through a lumen.

FIG. 8 is a perspective view illustrating some of the components of a localization system, according to some embodiments.

FIG. 9 is an end view illustrating the localization system in the loop, according to some embodiments.

FIG. 10 is an enlarged partial cross-sectional view, taken at area 10 of FIG. 9, illustrating layers of the localization system in the region of the proximal junction of the catheter.

FIG. 11 is a cross-sectional view illustrating components of the localization system in the region of the proximal junction of the catheter, taken at line 11-11 of FIG. 9, looking proximally towards the proximal junction.

FIG. 12 is a cross-sectional view illustrating components of the localization system in the intermediate loop section of the loop, taken at line 12-12 of FIG. 9.

FIG. 13 is a cross-sectional view illustrating components of the localization system in the distal loop section, taken at line 13-13 of FIG. 9.

FIG. 14 is an enlarged side view of FIG. 13 illustrating the attachment of an EM sensor to a distal shaft, according to some embodiments.

FIG. 15 is another cross-sectional view illustrating components of the localization system in the distal loop section, taken at line 15-15 of FIG. 9.

FIG. 16 is an end view of the distal face of the loop of an electrophysiology catheter, according to some embodiments.

FIG. 17 is a partial cross-sectional view of the tip taken at line 17 of FIG. 16.

### DETAILED DESCRIPTION

The present invention relates generally to a multi-lumen electrophysiology EP catheter. In particular, the multi-lumen catheter includes a localization/navigation system that is positioned in a first lumen and an electrode system including a first bundle of wires positioned in a second lumen and a second bundle of wires positioned in a third lumen. The first bundle of wires is connected to a first plurality of electrodes located at a distal end of the catheter, and the second bundle of wires is connected to a second plurality of electrodes located at the distal end of the catheter. The first bundle of wires and the second bundle of wires are electrically isolated from one another—as well as from the localization system—by virtue of placement in the respective first, second, and third lumens. The localization system positioned in the first lumen includes an electromagnetic (EM) sensor and EM sensor wires extending proximally through the catheter from the EM sensor. In some embodiments, the EM sensor wires are positioned within the first lumen to be located along a neutral axis of the multi-lumen catheter in order to reduce the force on the EM sensor wires during bending of the multi-lumen catheter.

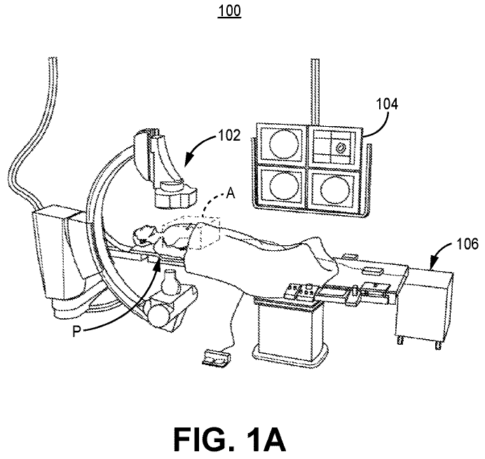

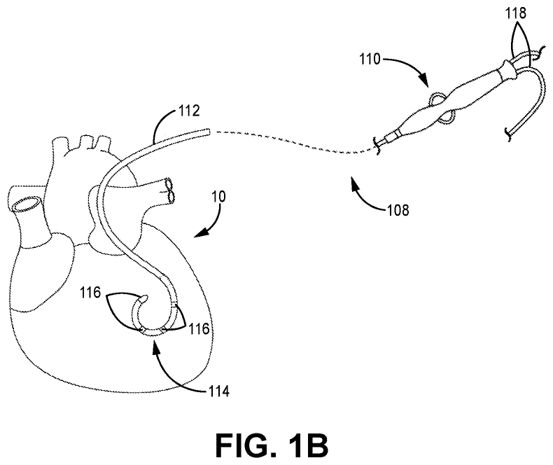

FIG. 1A and FIG. 1B are diagrammatic views of a treatment system 100 for providing treatment to area A of a patient P, according to some embodiments. As shown in FIG. 1B, the treatment area may be the heart 10. The treatment system 100 includes a magnetic transmitter assembly 102, a display 104, a control system 106 with at least one processing/control unit, and an electrophysiology catheter 108. The catheter 108 includes a handle 110 and a multi-lumen shaft 112 that extends distally from the handle 110. The catheter 108 includes a loop 114 with a plurality of electrodes 116. The electrodes 116 form a part of an electrode system configured for mapping and/or ablation. The catheter 108 includes at least one cable 118 to connect the catheter 108 to the control system 106. The catheter 108 includes a localization/navigation system that may be used with the magnetic transmitter assembly 102 to track/navigate the catheter 108. In at least one embodiment, the location system is positioned and/or extends along the neutral axis of the catheter 108.

In some embodiments, the control system 106 includes storage capable of storing computer readable instructions and an electronic control unit capable of executing computer readable instructions. In some embodiments, the control system 106 communicates bi-directionally and further displays information via display 104. In some embodiments, the control system 106 includes a generator configured to deliver electrical pulses to the electrodes 116. The amplitude (i.e., intensity), duration, and number of pulses may be controlled. For example, high voltage pulses may be delivered to the electrodes 116.

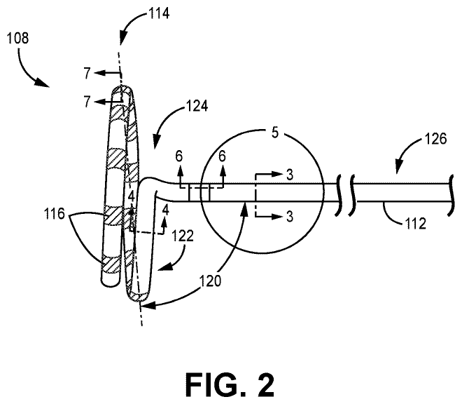

FIG. 2 is a side view of an electrophysiology catheter 108, according to some embodiments. In the embodiment shown in FIG. 2, the catheter 108 includes a multi-lumen shaft 112 that extends from the handle 110 at a proximal end to a distal end of the catheter 108. The catheter 108 includes a loop 114, a proximal junction 122, a transition region 124, and an elongate proximal region 126. The loop 114 is located at the distal end of the catheter 108. The loop 114 is connected to the transition region 124 via the proximal junction 122. In some embodiments, the transition region 124 provides a transition between the curvature of the loop 114 and the elongate proximal region 126. In these embodiments, the transition region 124 may be described as having a curved distal section and an elongate proximal section. The transition region 124 is in turn connected to the elongate proximal region 126 that extends proximally to a catheter handle 110 (shown in FIG. 1B). The loop 114 includes a plurality of electrodes 116. In some embodiments, the electrodes 116 are band electrodes extending around the circumference of the multi-lumen shaft 112. In some embodiments, the loop 114 may be adjustable and may be actuated from an undeployed state (utilized to navigate the loop to a desired region within the heart) to a deployed state as shown in FIG. 2. The loop 114 is at an angle 120 to the multi-lumen shaft 112. In at least one embodiment, the angle 120 may be approximately 80° to approximately 100°. In some embodiments, the angle 120 is at most 80°. In other embodiments, the angle 120 is at least 80°. In one example, angle 120 is approximately 90°.

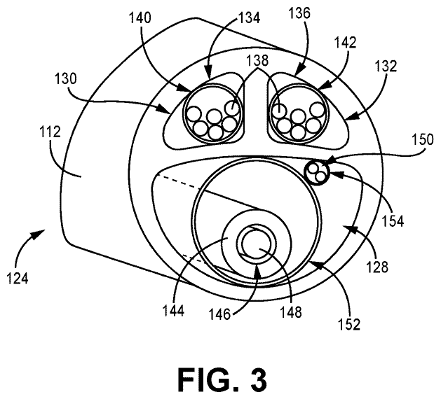

FIG. 3 is a cross-sectional view, taken in the transition region 124 of the catheter 108 at line 3-3 of FIG. 2, that illustrates the lumens comprising the multi-lumen shaft 112 according to some embodiments. In some embodiments, the multi-lumen shaft 112 includes a first lumen 128, a second lumen 130, and a third lumen 132. In some embodiments, the first lumen 128 houses components associated with the localization/navigation system, including a proximal shaft 144, a proximal shaft lumen 146, an activation wire 148, electromagnetic (EM) sensor wires 150, a heat shrink tube 152, and a heat shrink tube 154. In some embodiments, the second lumen 130 houses components associated with a first electrode system, including a first (even) electrode wire bundle 134 surrounded by the heat shrink tube 140. In some embodiments, the third lumen 132 houses components associated with the second electrode system, including a second (odd) electrode wire bundle 136 surrounded by the heat shrink tube 142.

In this exemplary configuration, the first lumen 128 is located in one half of the multi-lumen shaft 112, and the second and third lumens 130, 132 are located in the other half of the multi-lumen shaft 112. In some embodiments, the first lumen 128 is significantly larger than the second and third lumens 130, 132 (e.g., the first lumen 128 is twice as large as the second lumen 130). In some embodiments, components of the localization system are housed in and/or extend through the first lumen 128, and components of the electrode system are housed in and/or extend through the second and third lumens 130, 132. In one aspect, the components of the location system housed within the first lumen 128 are electrically isolated from the components of the electrode system housed in the second and third lumens 130, 132. Likewise, the first electrode wire bundle 134 located in the second lumen 130 is electrically isolated from the second electrode wire bundle 136 located in the third lumen 132. For example, in some embodiments, the material used to form the multi-lumen shaft 112 and that separates the lumens from one another is formed of a dielectric material. The dielectric material may have a minimum dielectric strength that is in excess of the operating voltage of the system to provide electrical isolation between components located in different lumens.

With respect to the location system located in the first lumen 128, an activation wire 148—extending from the handle 110 located at the proximal end—extends through a proximal shaft 144. In one example, the proximal shaft 144 is a metal coil shaft. The proximal shaft 144 includes a proximal shaft lumen 146. In some embodiments, the activation wire 148 is housed in/extends through the proximal shaft lumen 146. A first heat shrink tube 152 may surround the proximal shaft 144. In other words, the first heat shrink tube 152 is on the outer surface of the proximal shaft 144. The first heat shrink tube 152 may be described as forming an outer layer of the proximal shaft 144. In some embodiments, the activation wire 148 is connected at the distal end of the loop and is actuated via the handle 110 to change the geometry of the loop 114.

In some embodiments, EM sensor wires 150 extend from the handle 110 on a proximal end to one or more EM sensors. As described in more detail below, EM sensors are utilized to detect/measure magnetic fields, wherein the measured fields are utilized to determine the location of the EM sensor. In some embodiments, EM sensors may be located at one or more of a plurality of locations, including proximal to the loop 114, within the loop 114, or at a distal end of the loop 114. In some embodiments, the EM sensor wires 150 extend alongside the proximal shaft 144 and/or heat shrink tube 152. In some embodiments, a heat shrink tube 154 surrounds the EM sensor wires 150 in the transition region 124. The heat shrink tube 154 may provide additional electrical isolation between the localization system and the electrode system located in the second and third lumens 130, 132. For example, in some embodiments the heat shrink tube 154 is a Polyethylene Terephthalate (PET), which is a high dielectric insulation material. In some embodiments, the EM sensor wires 150 are located along a neutral axis of the catheter 108 (approximately the middle of the multi-lumen shaft 112).

In the embodiment shown in FIG. 3, each electrode wire bundle 134, 136 includes a plurality of electrode wires 138 surrounded by the heat shrink tubes 140, 142, respectively. In some embodiments, the heat shrink tubes 140, 142 provide additional electrical isolation between the electrode system and the localization system. First (even) electrode wire bundle 134 includes wires that are connected to one or more of a first group of electrodes (e.g., a first plurality of electrodes 116 located on loop 114). Second (odd) electrode wire bundle 136 includes wires that are connected to one or more of a second group of electrodes (e.g., a second plurality of electrodes 116 located on loop 114). In some embodiments, first and second electrode wire bundles 134, 136 are utilized to carry opposite polarity voltages utilized in ablation processes (e.g., pulsed field ablation). Insulation between the first and second group of electrode wire bundles 134, 136 is therefore also important to prevent shorts between the respective wire bundles. In some embodiments, the portion of the multi-lumen shaft 112 located between the second and third lumens 130, 132 provides insulation between the respective wire bundles 134, 136. In some embodiments, heat shrink tubes 140, 142 also act to provide additional insulation between the respective wire bundles 134, 136. As described above, heat shrink tubes 140, 142 may also be comprised of a PET material.

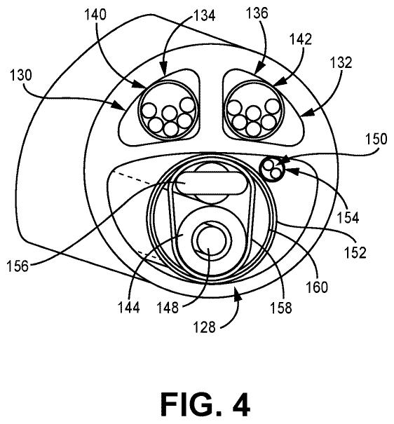

FIG. 4 is another cross-sectional view of the transition region 124 of the catheter 108, taken at line 4-4 of FIG. 2. As shown in FIG. 3, the multi-lumen shaft 112 includes first, second, and third lumens 128, 130, 132. In contrast with the cross-sectional view taken in the linear portion of the transition region 124 as shown in FIG. 3, catheter components in this section of the transition region 124 further includes a shape wire 156 extending alongside the proximal shaft 144. In some embodiments, the shape wire 156 extends distally from a location within the transition region 124 to the distal end of the multi-lumen shaft 112. The shape wire 156 may be formed of a shape memory material configured to provide the loop 114 with its shape. In some embodiments, the shape memory material is a metal. In one example, the shape memory material is nitinol. In some embodiments, an additional heat shrink tube 158 secures the activation wire 148 and the shape wire 156 together. In additional embodiments, a third heat shrink tube 160 is interposed between heat shrink tubes 152, 158. In these embodiments, the proximal shaft 144 and the activation wire 148 are covered by a first (inner) layer formed by the heat shrink tube 158, a second (middle) layer formed by the heat shrink tube 160, and the heat shrink tube 152. The heat shrink tubes 152, 158, and 160 may comprise the same or different materials. For example, the heat shrink tubes 152, 158, and 160 may be comprised of a PET material.

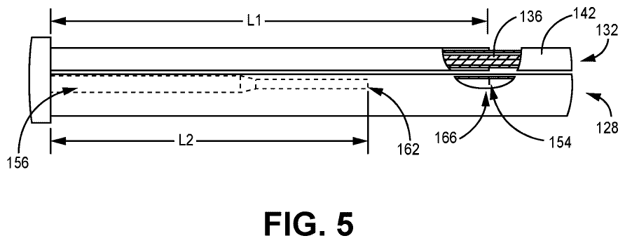

FIG. 5 is an enlarged view of area 5 of FIG. 2 with a portion of the multi-lumen shaft 112 in the transition region 124 removed to show the location system and the electrode system, according to some embodiments. The proximal end 166 of the transition region 124 is shown on the right side, and is illustrated by length L1, which extends from the most distal portion shown to the proximal end 166 of the transition region 124. In addition, the proximal end 162 of shape wire 156 is shown, extending from a position noted by length L2 distally toward the distal end of the transition region 124. The proximal end 162 of the shape wire 156 is distal to the proximal end 166 of the transition region 124. In this example, the proximal end region of the shape wire 156 has a different diameter and/or shape than the rest of the shape wire 156. The proximal end of the heat shrink tube 154 is coextensive with the proximal end 166 of the transition region 124. In some embodiments, in the portion of the multi-lumen shaft extending proximal of the transition region the EM sensor wires 150 are free-floating (i.e., not surrounded by heat shrink tubes or otherwise affixed to adjacent structures). The heat shrink tube 142 is partially removed on the proximal end to illustrate the second electrode wire bundle 136 located in the third lumen 132. A portion of the heat shrink tubes 158, 160 surrounding the activation wire 148 are removed to illustrate the heat shrink tube 154 surrounding the EM sensor wires 150. The proximal end of heat shrink tube 154 is coextensive with the proximal end 166 of the transition region 124. That is, the EM sensor wires 150 extending proximal of the proximal end 166 of the transition region 124 are not surrounded by heat shrink.

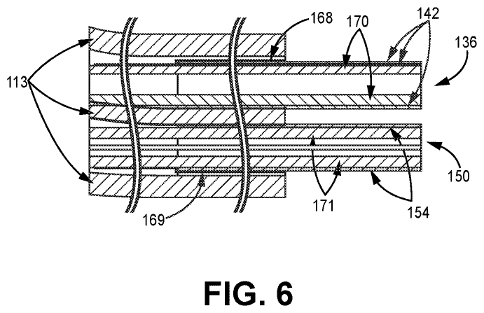

FIG. 6 is a cross-sectional view, taken at line 6-6 in FIG. 2, illustrating the proximal junction 122 an area where the loop 114 and the transition region 124 abut. Visible in this cross-sectional view are portions of the walls 113 of the multi-lumen catheter 112, the electrode wire bundle 136, the EM sensor wires 150, and adhesive 168, 169, 170, and 171. In some embodiments, the adhesive 170 surrounds the electrode wire bundle 136 within the heat shrink tube 142. Likewise, in some embodiments, the adhesive 171 surrounds the EM sensor wires 150 within the heat shrink tube 142. In some embodiments, the adhesive may provide additional insulation between the respective electrode wire bundles 136 and 134 and between the respective wire bundles 134, 136 and the EM sensor wires 150. In some embodiments, an adhesive/epoxy 168 is utilized to secure an outer surface of the heat shrink tube 142 to the inner surface of the multi-lumen shaft 112. Likewise, adhesive/epoxy 169 is utilized to secure an outer surface of the heat shrink tube 154 to the inner surface of the multi-lumen shaft 112. In some embodiments, the adhesive/epoxy 168, 169 are applied at the proximal junction 122. In some embodiments, the adhesive/epoxy 168, 169 are applied at the proximal junction 122 and continue forward in the distal direction.

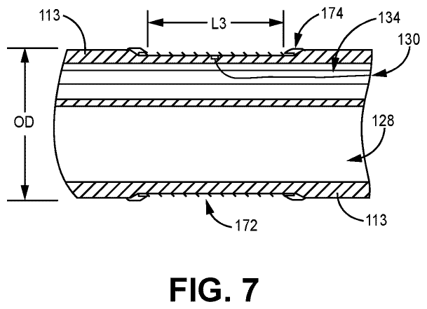

FIG. 7 is a cross-sectional view of the loop 114, taken at line 7-7 of FIG. 2, illustrating a portion of the electrode system. For clarity, the components of the localization system in the first lumen 128 are not included. The multi-lumen shaft 112 has an outer diameter OD. In this example, the multi-lumen shaft 112 defines a recess 172 formed within the wall 113, with a width L3, for a band electrode 116 (not shown). Adhesive 174 may be used to secure the electrode 116 to the recess 172 and/or the multi-lumen shaft 112. In some embodiments, the adhesive 174 is a circle or band of epoxy. In the embodiment shown in FIG. 7, first (even) electrode wire bundle 134 is shown within the second lumen 130, with at least one of the wires included as part of the wire bundle 134 being brought into contact with the electrode 116 secured within the recess 172. The at least one electrode wire extends through the wall 113 of multi-lumen shaft 112 to form an electrical connection with the electrode 116.

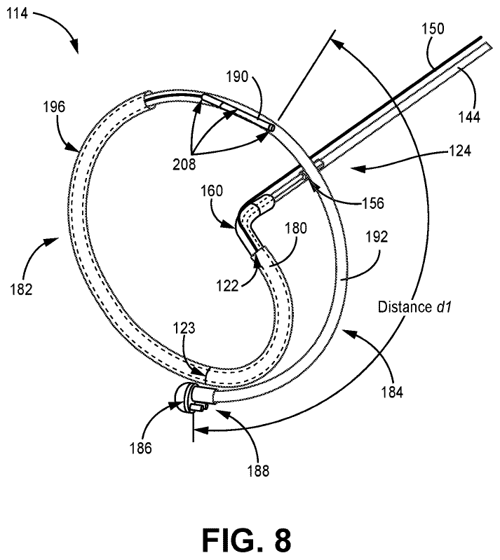

FIG. 8 is a perspective view of the catheter 108 with the multi-lumen shaft 112 and the electrode system removed to highlight the components of the localization system according to some embodiments. In particular, the transition region 124 and the loop 114 of the localization system are illustrated along with removal of certain layers (e.g., heat shrink tube 196) along portions of the loop 114 to illustrate the location of the EM sensor 190. As described above, the transition region 124 includes a linear section and a curved section. The proximal end of the linear section is connected to the elongate proximal region that extends to the handle 110 (as shown in FIGS. 1 and 2). The distal end of the curved section abuts the loop 114 at the proximal junction 122. The loop 114 includes a proximal loop section 180, an intermediate loop section 182, and a distal loop section 184.

In the transition region 124 shown in FIG. 8, the proximal shaft 144 extends distally from the linear section of the transition region 124 to the curved section of the transition region 124. The EM sensor wires 150—twisted in this embodiment—extend alongside the proximal shaft. The proximal end 162 of the shape wire 156 is shown starting in the linear section of the proximal shaft 144 and extending distally toward the curved section. In addition, the heat shrink tube 160 is shown surrounding the proximal shaft 144 and the shape wire 156. In some embodiments, the EM sensor wire 150 remains external to the heat shrink tube 160, which only extends along the curved section of the transition region 124. In some embodiments, the heat shrink tube 160 ends proximal to or approximately equal to the proximal junction 122. As described in more detail below, the proximal junction 122 corresponds with the distal end of the proximal shaft 144 abutting with the proximal end of the distal shaft 192.

In some embodiments, the heat shrink tube 196 has a proximal end that corresponds with the proximal junction 122. As described in more detail below, the heat shrink tube 196 extends distally from a proximal junction 122 to the boundary between the intermediate loop section 182 and the distal loop section 184. In some embodiments, the heat shrink tube 196 extends over the EM sensor wires 150, distal shaft 192, shape wire 156, and activation wire 148.

The loop 114 is shown extending distally from the proximal junction 122 to the tip 186 located at the distal end of the catheter 108. In some embodiments, the tip 186 may be atraumatic. For example, the distal end of the tip 186 may be rounded. The tip 186 may include at least one post 188, located along the outer side of the loop 114. In some embodiments, the tip 186 is a molded component. As described above, the loop 114 is divided into a plurality of sections, including a proximal loop section 180, an intermediate loop section 182, and a distal loop section 184. The proximal loop section 180 extends distally from the proximal junction 122. The intermediate loop section 182 extends distally from a distal end 123 of the proximal loop section 180, and the distal loop section 184 extends distally from the intermediate loop section 182.

In some embodiments, the location system includes at least one EM sensor 190 configured to detect and measure the presence of a magnetic field and communicate a signal (e.g., voltage) representative of the measured magnetic field to the handle 110. In some embodiments, the EM sensor 190 is comprised of one or more coils, either open-core coils or magnetic-core coils. In some embodiments, one or more EM sensor 190 are affixed to the loop 114 to provide location/orientation information in the presence of a magnetic field. In the embodiment shown in FIG. 8, the EM sensor 190 is located in the distal loop section 184, generally opposite to the distal tip 186. In other embodiments, the EM sensor 190 could be located in the proximal loop section 180, the intermediate loop section 182, and/or the distal tip 186 located at the distal end of the loop 114. As described above, the EM sensor wires 150 may be located along a neutral axis of the multi-lumen shaft 112 (e.g., located close to the center of the multi-lumen shaft 112 as shown in FIGS. 3 and 4). As the loop 114 is deployed from the relatively straight geometry to the curved geometry shown in FIG. 8, the EM sensor wires will be subjected to less stress when located along the neutral axis of the multi-lumen shaft 112.

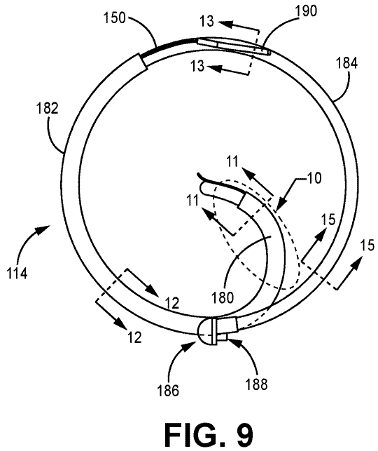

FIG. 9 is an end view of the loop 114 with the multi-lumen shaft 112 and the electrode system removed to highlight the components of the localization system, according to some embodiments. In the embodiment shown in FIG. 8, cross-sections are taken at various locations with respect to the proximal junction 122 and various portions of the loop 114 to illustrate the structure of the localization systems through the distal end of the catheter 108. For example, a longitudinal cross-section is taken at cut 10 and a cross-section taken at 11-11 illustrate those components provided in the region of the proximal junction 122 and the proximal loop section 180. Cross-sectional view taken at 12-12 illustrates the components of the localization system included within the intermediate loop section 182. Cross-sectional view taken at 13-13 illustrates the affixing of the EM sensor 190 to the distal shaft 192, and cross-sectional view taken at 15-15 illustrates components included in the distal loop section 184 (e.g., distal of the EM sensor 190).

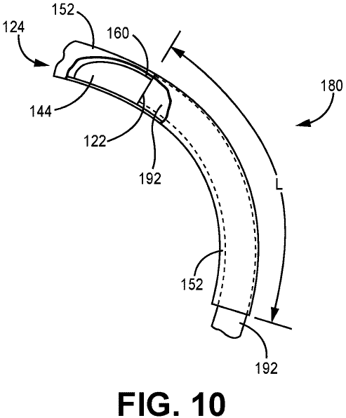

FIG. 10 is an enlarged partial cross-sectional view, taken at line 9 of FIG. 9, illustrating layers of the localization system located within the first lumen 128 at the proximal loop section 180. As shown in FIG. 10, the proximal loop section 180 abuts the transition region 124 at the proximal junction 122. On the proximal side of the proximal junction 122, the location system includes the heat shrink tubes 152 and 160 surrounding the proximal shaft 144. On the distal side of the proximal junction 122, the localization system includes the heat shrink tubes 152 and 160 surrounding a distal shaft 192. In some embodiments, the proximal shaft 144 abuts the distal shaft 192. In some embodiments, the distal shaft 192 is a braided polyimide tube capable of handling stress associated with changes in shape of the loop 114.

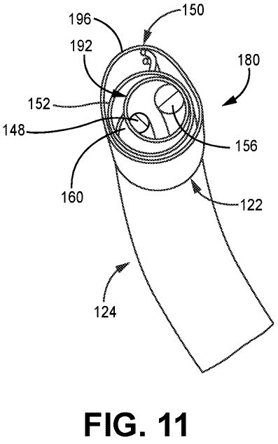

FIG. 11 is a cross-sectional view illustrating components of the localization system in the region of the proximal junction 122 of the catheter 108, taken at line 10-10 of FIG. 9, looking proximally towards the proximal junction 122. In this view, the transition region 124 extends downward from the proximal junction 122 and the proximal loop section 180 extends upward from the proximal junction 122. As described with respect to FIG. 10, the distal shaft 192 abuts the proximal shaft 144. In some embodiments, the proximal shaft 144 surrounded the activation wire 148, but did not encompass or surround the shape wire 156. In some embodiments, the distal shaft 192 surrounds both the activation wire 148 and the shape wire 156 (e.g., the activation wire 148 and the shape wire 156 are disposed within the lumen of the distal shaft 192). As described above, in some embodiments the distal shaft 192 is a braided polyimide shaft. Furthermore, in some embodiments, the heat shrink tube 152 surrounds the distal shaft 192.

In some embodiments, EM sensor wires 150 extend along an outer surface of the heat shrink tube 152. In addition, in some embodiments, the heat shrink tube 196 extends distally from the proximal junction 122 and surrounds the EM sensor wires 150 as well as the heat shrink tube 152. As described above, in some embodiments it is beneficial to place the EM sensor wires 150 along a neutral axis within the catheter 108. In order for the EM sensor wires 150 to be located along a neutral axis with respect to the entire multi-lumen catheter 108, the EM sensor wires 150 may be located on one side of the distal shaft 192 (and corresponding heat shrink tube 152) within the first lumen 128 (i.e., not centrally located within the first lumen 128).

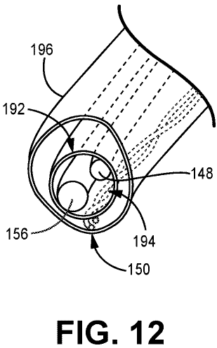

FIG. 12 is a cross-sectional view illustrating components of the localization system in the intermediate loop section 182 of the loop 114, taken at line 12-12 of FIG. 9. At this portion of the loop, the activation wire 148 and the shape wire 156 are located within the lumen 194 of the distal shaft 192. In some embodiments, the heat shrink tube 152 does not extend into the intermediate loop section 182. In this embodiment, the EM sensor wires 150 are located between the distal shaft 192 and the heat shrink tube 196. In other embodiments, the heat shrink tube 152 may extend into the intermediate loop section 182, depending on the location of the EM sensor 190. In some embodiments, one or more EM sensors 190 are located in the intermediate loop section 182, in the distal loop section 184 (as shown in FIG. 12), and/or at the distal end of the loop 114 adjacent to or part of the distal tip 186. In some embodiments, if a plurality of EM sensors 190 are utilized either proximal to the loop 114, within the loop 114, or at the distal tip 186 of the loop 114 then a plurality of the EM sensor wires 150 may be required.

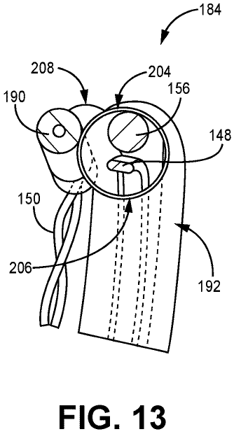

FIG. 13 is a cross-sectional view illustrating components of the localization system in the distal loop section 184, taken at line 13-13 of FIG. 9. In the embodiment shown in FIG. 12, the EM sensor 190 is located in the distal loop section 184 of the loop 114. The EM sensor wires 150 are shown connected to the EM sensor 190. To ensure the position of the EM sensor 190 remains fixed relative to the distal shaft 192, the EM sensor 190 is affixed to the outer surface of the distal shaft 192. In some embodiments, the EM sensor 190 is affixed to the outer surface via a gel adhesive 208. The EM sensor 190 may be comprised of one or more coils configured to detect magnetic fields. The coils may be wrapped around a magnetic core or may be open-core coils. In some embodiments, the EM sensor 190 includes a magnetic core. The one or more coils generate a voltage proportional to the magnitude of the detected field. The generated voltage is communicated to the handle 110 via the EM sensor wires 150. In embodiments in which an additional EM sensor is located at the distal tip 186 of the loop 114 (or at any location distal to the location of EM sensor 190) then additional EM sensor wires 150 would extend distally beyond the EM sensor 190.

As discussed above, the shape wire 156 and the activation wire 148 are located within the lumen defined by/included in the distal shaft 192. In some embodiments, the shape wire 156 is located closer (relative to the activation wire 148) to the outer side 204 of the loop 214. The activation wire 148 is located closer (relative to the shape wire 156) to the inner side 206 of the loop 214.

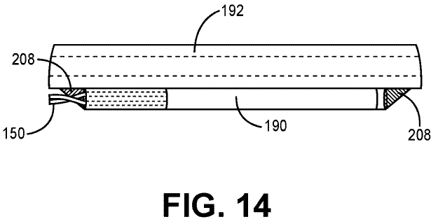

FIG. 14 is an enlarged side view of FIG. 13 illustrating the attachment of an EM sensor to a distal shaft, according to some embodiments. As discussed above, the EM sensor 190 is affixed to the distal shaft 192. In some embodiments, the EM sensor 190 and at a least a portion of adjacent EM sensor wires 150 are affixed to the distal shaft 192 via the gel adhesive 208.

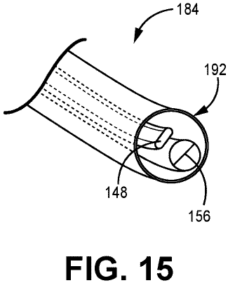

FIG. 15 is another cross-sectional view illustrating components of the localization system in the distal loop section 184, taken at line 15-15 of FIG. 9. As described above, activation wire 148 and shape wire 156 are positioned within the lumen of the distal shaft 192. The view shown in FIG. 15 is taken distal to the location of the EM sensor 190, and therefore does not include an EM sensor and/or EM sensor wires. In other embodiments in which the EM sensor 190 is positioned at the distal tip 186 of the loop 114, the EM sensor wires 150 may extend along an outer surface of the distal shaft 192. As described above, in some embodiments the distal shaft is comprised of a braided polyimide (PI) material. In some embodiments, the shape wire 156 is comprised of a nitinol material that includes shape memory properties.

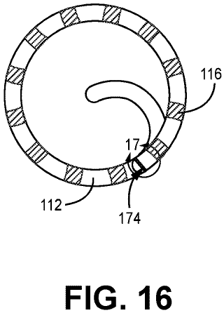

FIG. 16 is an end view of the loop 114, including the multi-lumen shaft 112 and the plurality of electrodes 116 affixed to an outer surface or within recesses 172 (shown in FIG. 7) of the multi-lumen shaft 112. In some embodiments, the electrodes 116 are utilized for one or more functions, including impedance measurements for localization, electrophysiological mapping, delivery of ablation energy (e.g., pulsed field ablation). In some embodiments, the electrodes 116 each have the same geometry. In other embodiments, the electrodes 116 may include geometries that vary with the operation or function performed by the electrode (e.g., ablation electrodes may have larger geometries than electrodes utilized for impedance measurement). In some embodiments, the electrodes 116 are evenly spaced along the length of the loop 114. In other embodiments, spacing of the electrodes may depend on the function performed by the electrodes. In some embodiments, a first plurality of the electrodes 116 are designated as “even” electrodes connected to the wires included as part of the first (even) wire bundle 134 (shown in FIGS. 3 and 4). Likewise, a second plurality of the electrodes 116 are designated as “odd” electrodes connected to the wires included as part of the second (odd) wire bundle 136.

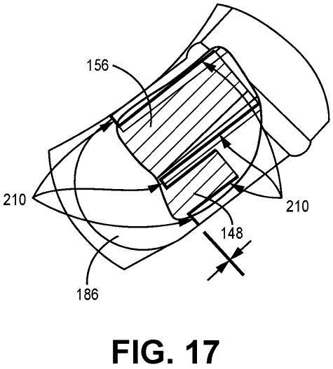

FIG. 17 is a partial cross-sectional view of the tip taken at line 17 of FIG. 16. In the embodiment shown in FIG. 17, the shape wire 156 and the activation wire 148 terminate at the distal tip 186. In some embodiments, the shape wire 156 is affixed to the distal tip 186 via an epoxy 210 that at least partially surrounds the shape wire 156. Likewise, in some embodiments the activation wire 148 is affixed to the distal tip 186 via an epoxy that at least partially surrounds the activation wire 148. In this way, the activation wire 148 and the shape wire 156 are affixed to the distal tip 186. In particular, a longitudinal force exerted in a proximal direction by the activation wire results in the circumference/geometry of the loop 114 being modified without the activation wire 148 becoming disengaged from the distal tip 186.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A catheter comprising:
a multi-lumen catheter shaft having a proximal end, a transition region, and a loop extending from a distal end of the transition region to a distal tip, the catheter shaft comprising a first lumen, a second lumen, and a third lumen;
a location system positioned in the first lumen, the location system including a distal shaft that extends within the first lumen along at least a portion of the loop, an electromagnetic (EM) sensor attached to an outer surface of the distal shaft at a location along the loop, and a pair of electromagnetic (EM) sensor wires extending proximally from the EM sensor to a proximal end of the catheter shaft, the EM sensor and the pair of EM sensor wires positioned along a neutral axis of the catheter shaft within the first lumen; and
an electrode system including a first plurality of electrodes positioned along an outer surface of the loop, a second plurality of electrodes positioned along the outer surface of the loop, a first plurality of wires connected to the first plurality of electrodes and extending through the second lumen, and a second plurality of wires connected to the second plurality of electrodes and extending through the third lumen.

2. The catheter of claim 1, wherein the location system is electrically isolated from the electrode system.

3. The catheter of claim 1, wherein the EM sensor is affixed by adhesive to the outer surface of the distal shaft.

4. The catheter of claim 1, wherein the EM sensor wires are free-floating along an outer surface of the distal shaft.

5. The catheter of claim 4, further comprising:
a shape wire affixed to the distal tip and extending proximally from the distal tip through a lumen included within the distal shaft; and
an activation wire affixed to the distal tip and extending proximally from the distal tip through the lumen included within the distal shaft.

6. The catheter of claim 5, wherein the loop includes a proximal loop section extending distally from the transition region, an intermediate loop section extending distally from the proximal loop section, and a distal loop section extending distally from the intermediate loop section, wherein the EM sensor is located in the distal loop section and wherein the EM sensor wires are free-floating along an outer surface of the distal shaft.

7. The catheter of claim 6, further comprising:
a first heat shrink tube located in the intermediate loop section that secures the EM sensor wires to the outer surface of the distal shaft in at least the intermediate loop section of the loop.

8. The catheter of claim 7, further comprising:
a second heat shrink tube located in the proximal loop section that surrounds the distal shaft, wherein the EM sensor wires extends between the first heat shrink tube and the second heat shrink tube.

9. The catheter of claim 8, further comprising:
a proximal junction located between the loop and the transition region, wherein the distal shaft extends distally from the proximal junction and a proximal shaft extends proximally from the proximal junction, wherein the activation wire is located within a lumen of the proximal shaft and wherein the shape wire is located external to the proximal shaft.

10. The catheter of claim 9, wherein a distal end of the proximal shaft abuts a proximal end of the distal shaft at the proximal junction.

11. The catheter of claim 9, further including a third heat shrink tube surrounding the EM sensor wires and extending proximally from the proximal junction, wherein the third heat shrink tube extends along an outer surface of the second heat shrink tube.

12. The catheter of claim 11, further including a fourth heat shrink tube surrounding the proximal shaft and the shape wire in at least a portion of the transition region.

13. The catheter of claim 9, wherein a fifth heat shrink tube surrounds the first plurality of wires located within the second lumen and a sixth heat shrink tube surrounds the second plurality of wires located within the third lumen.

14. The catheter of claim 1, wherein the second and third lumens of the multi-lumen shaft are approximately equal in size and wherein the first lumen is larger than the second lumen and the third lumen.

15. An electrophysiology catheter comprising:
a multi-lumen shaft having a proximal end, a transition region, and a loop extending from a distal end of the transition region to a distal tip, the multi-lumen shaft comprising a first lumen, a second lumen, and a third lumen;
a localization system positioned in the first lumen, the localization system including an electromagnetic (EM) sensor and a pair of electromagnetic (EM) sensor wires extending proximally from the EM sensor; and
an electrode system positioned in the first and second electrode lumens, the electrode system including a first plurality of electrode wires extending through the second lumen and connected to a first plurality of electrodes located on an outer surface of the loop and a second plurality of electrode wires extending through the third lumen and connected to a second plurality of electrodes located on the outer surface of the loop, wherein the localization system is electrically isolated from the electrode system by material forming the multi-lumen shaft.

16. The catheter of claim 15, wherein the EM sensor is positioned on, and the pair of EM sensor wires extend along, a neutral axis of the multi-lumen shaft.

17. The catheter of claim 15, wherein the localization system includes a proximal shaft and a distal shaft located within the first lumen, wherein the proximal shaft extends proximally from a proximal junction defined between the transition region and the loop and the distal shaft extends distally from the proximal junction, wherein the EM sensor is affixed to the distal shaft.

18. The catheter of claim 17, wherein the localization system further includes a shape wire extending from transition region to the distal tip, wherein the shape wire extends within a lumen included within the distal shaft and extends along an outer surface of the proximal shaft.

19. The catheter of claim 18, wherein the localization system further includes an activation wire for modifying a geometry of the loop, the activation wire extending from the proximal catheter end to the distal tip, wherein the activation wire extends within a lumen of the proximal shaft and within the lumen of the distal shaft.

20. The catheter of claim 15, the loop comprising a proximal loop section, an intermediate loop section, and a distal loop section, wherein the EM sensor is positioned in the distal loop section, wherein EM sensor wires surrounded by a first heat shrink tube in the transition region, secured between a second heat shrink tube and a distal shaft in the proximal and intermediate loop sections, and are free floating in the distal loop section.
